(19) United States
(12) Patent Application Publication
(10) Pub. No.: US 2020/0353163 A1
(43) Pub. Date: Nov. 12, 2020

(54) SYRINGE PUMP SYSTEM AND METHOD FOR OPERATING THE SAME

(71) Applicants: University of South Carolina, Columbia, SC (US); Prisma Health, Columbia, SC (US)

(72) Inventor: William Hand, Columbia, SC (US)

(21) Appl. No.: 16/868,724

(22) Filed: May 7, 2020

Related U.S. Application Data
(60) Provisional application No. 62/844,290, filed on May 7, 2019.

(51) Int. Cl. A61M 5/168; G16H 20/17

(52) U.S. Cl. CPC A61M 5/16804; G16H 20/17; A61M 2205/3306; G06K 7/1413; A61M 2205/502; A61M 2205/6072; A61M 2205/52

(57) ABSTRACT

A method for operating a syringe pump assembly includes receiving a syringe in an opening of a drug receptacle of a pump of the syringe pump assembly; determining information indicative of the contents of the syringe using a sensor positioned in the drug receptacle of the pump of the syringe pump assembly; receiving drug administration data from a user; determining an appropriateness of the received drug administration data view of the determined information indicative of the contents of the syringe and further in view of patient-specific information received from an electronic medical records system; and making an administration decision for the contents of the syringe based on the determined appropriateness of the received drug administration data.

### CROSS-REFERENCE TO RELATED APPLICATIONS

This application is a non-provisional application claiming the benefit of priority under 35 U.S.C. § 119(e) to U.S. Provisional Application No. 62/844,290, filed May 7, 2019, which is hereby incorporated by reference in its entirety.

### FIELD

The present subject matter relates generally to a syringe pump system and a method for operating the same.

### BACKGROUND

Within the field of anesthesiology, correctly administering various medications is critical. With current administration protocols, it is possible for errors to occur. For example, the medication may be administered in the wrong order, at the wrong rate, in the wrong amount, etc. Causes of drug errors are wide-ranging and implicate systems, packaging, and human error. Each of these errors may have negative consequences for the patient being treated.

In at least certain situations, pre-filled and labeled syringes may be used. Such pre-filled and labeled syringes may improve the safety of IV drug administration for several reasons including: consistent labeling, decreased risk of contamination, prevention of dilution miscalculation, and (potentially) improved visual discrimination. However, human error (e.g. no-read-errors) continues to cause negative outcomes.

Accordingly, a system and method for further reducing a risk of these errors would be welcomed in the field.

### BRIEF DESCRIPTION OF THE INVENTION

Aspects and advantages of the invention will be set forth in part in the following description, or may be obvious from the description, or may be learned through practice of the invention.

In one exemplary aspect of the present disclosure, a method for operating a syringe pump assembly is provided. The method includes receiving a syringe in an opening of a drug receptacle of a pump of the syringe pump assembly; determining information indicative of the contents of the syringe using a sensor positioned in the drug receptacle of the pump of the syringe pump assembly; receiving drug administration data from a user; determining an appropriateness of the received drug administration data view of the determined information indicative of the contents of the syringe and further in view of patient-specific information received from an electronic medical records system; and making an administration decision for the contents of the syringe based on the determined appropriateness of the received drug administration data.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A disclosure of the present invention is set forth in the specification, which makes reference to the appended figures, in which:

FIG. 1 is a schematic view of an EMR-controlled syringe pump assembly in accordance with an exemplary embodiment of the present disclosure.

FIG. 2 is a schematic view of a drug receptacle of the EMR-controlled syringe pump assembly of FIG. 1.

FIG. 3 is a schematic, cross-sectional view of the drug receptacle of the EMR-controlled syringe pump assembly of FIG. 1, viewed along Line 3-3 in FIG. 2.

FIG. 4 is a flow diagram of a method of operating a syringe pump assembly in accordance with an exemplary aspect of the present disclosure.

### DETAILED DESCRIPTION

Reference will now be made in detail to present embodiments of the invention, one or more examples of which are illustrated in the accompanying drawings. The detailed description uses numerical and letter designations to refer to features in the drawings. Like or similar designations in the drawings and description have been used to refer to like or similar parts of the invention.

As used herein, the terms “first”, “second”, and “third” may be used interchangeably to distinguish one component from another and are not intended to signify location or importance of the individual components.

The terms “coupled,” “fixed,” “attached to,” and the like refer to both direct coupling, fixing, or attaching, as well as indirect coupling, fixing, or attaching through one or more intermediate components or features, unless otherwise specified herein.

The singular forms “a”, “an”, and “the” include plural references unless the context clearly dictates otherwise.

Approximating language, as used herein throughout the specification and claims, is applied to modify any quantitative representation that could permissibly vary without resulting in a change in the basic function to which it is related. Accordingly, a value modified by a term or terms, such as “about”, “approximately”, and “substantially”, are not to be limited to the precise value specified. In at least some instances, the approximating language may correspond to the precision of an instrument for measuring the value, or the precision of the methods or machines for constructing or manufacturing the components and/or systems. For example, the approximating language may refer to being within a 10 percent margin.

The present disclosure is generally related to a syringe pump assembly and system for reducing medication administration errors in, e.g., the perioperative clinical setting. The disclosed syringe pump assembly works with an electronic medical record system (“EMR”) to ensure the drugs are administered in a manner prescribed by the administrator (e.g., a doctor or nurse) and further in a manner consistent with the patient's profile, as determined by the EMR.

In at least one specific embodiment, the present disclosure relates to an EMR-controlled syringe pump assembly including a sensor embedded in a chamber housing for automatically reading certain information related to drug installed therein, such as a pre-filled and labeled syringe. As discussed more fully below, the sensor may be a barcode reader, a QR code reader, a different visual/graphical reader/sensor, a tactile sensor, electromagnetic sensor, or any other type of sensor capable of sensing or otherwise receiving data from a drug installed therein. The syringe pump may communicate the read/sensed drug information (as well as any doctor/nurse inputs regarding, e.g., administration information of the drug) to the EMR, which may cross-reference the information against patient-specific information in the EMR, known medical database information, etc. to determine if the drug and drug administration information is appropriate. If appropriate, the EMR may provide the syringe pump with instructions to proceed. It will be appreciated that the term “appropriate” as used in this application with respect to drug administration data for a particular patient refers to the drug administration data being suitable under the particular circumstances for the patient.

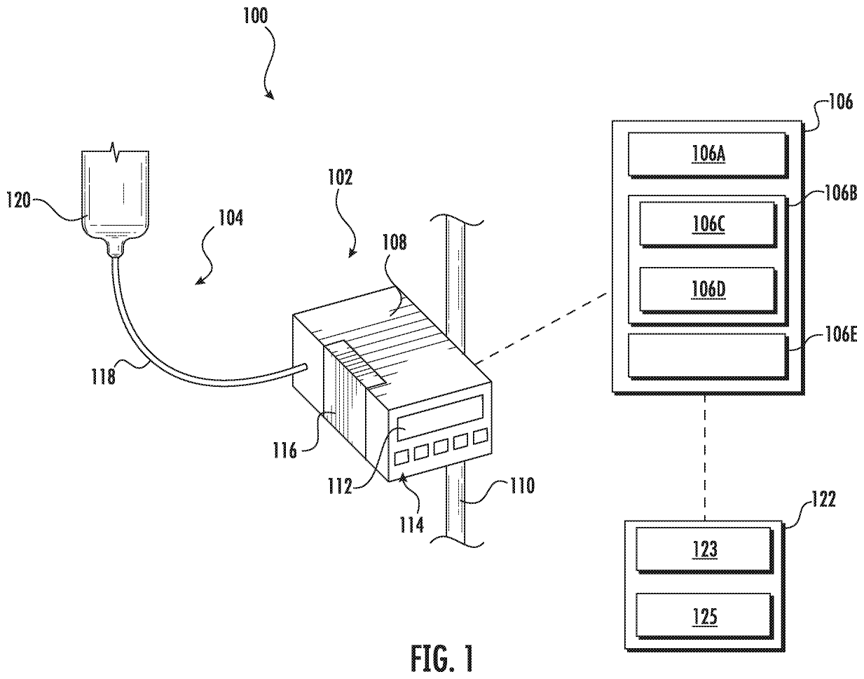

Referring now to the drawings, FIG. 1 shows an EMR-controlled syringe pump assembly 100 in accordance with the present disclosure. The syringe pump assembly 100 generally includes a pump 102, an output 104, and a controller 106. The pump 102 includes a body 108 coupled to a pole stand 110. The body 108 of the pump 102 includes a display 112 and a plurality of inputs 114. It will be appreciated, however, that in other embodiments, the body 108 of the pump 102 may be configured in any other suitable manner (including, e.g., any configuration of displays, inputs, outputs, etc.).

The body 108 of the pump 102 further includes a drug receptacle 116. As will be described in more detail below, the drug receptacle 116 is configured to receive one or more pre-filled and labeled syringes, and the pump 102 is further configured to provide the contents of the syringes to the output 104, in the manner described herein. For the embodiment shown, the output 104 includes a line 118 and an IV bag 120. The contents of the IV bag 120 may be provided to a patient when in use.

Moreover, the syringe pump assembly 100 includes the controller 106. The controller 106 is depicted as a separate component from the pump 102 in the embodiment shown. However, in other embodiments, the controller 106 may be integrated into the pump 102.

Referring particularly to the operation of the controller 106, in at least certain embodiments, the controller 106 can include can include one or more processor(s) 106A and one or more memory device(s) 106B. The one or more processor(s) 106A can include any suitable processing device, such as a microprocessor, microcontroller, integrated circuit, logic device, and/or other suitable processing device. The one or more memory device(s) 106B can include one or more computer-readable media, including, but not limited to, non-transitory computer-readable media, RAM, ROM, hard drives, flash drives, and/or other memory devices.

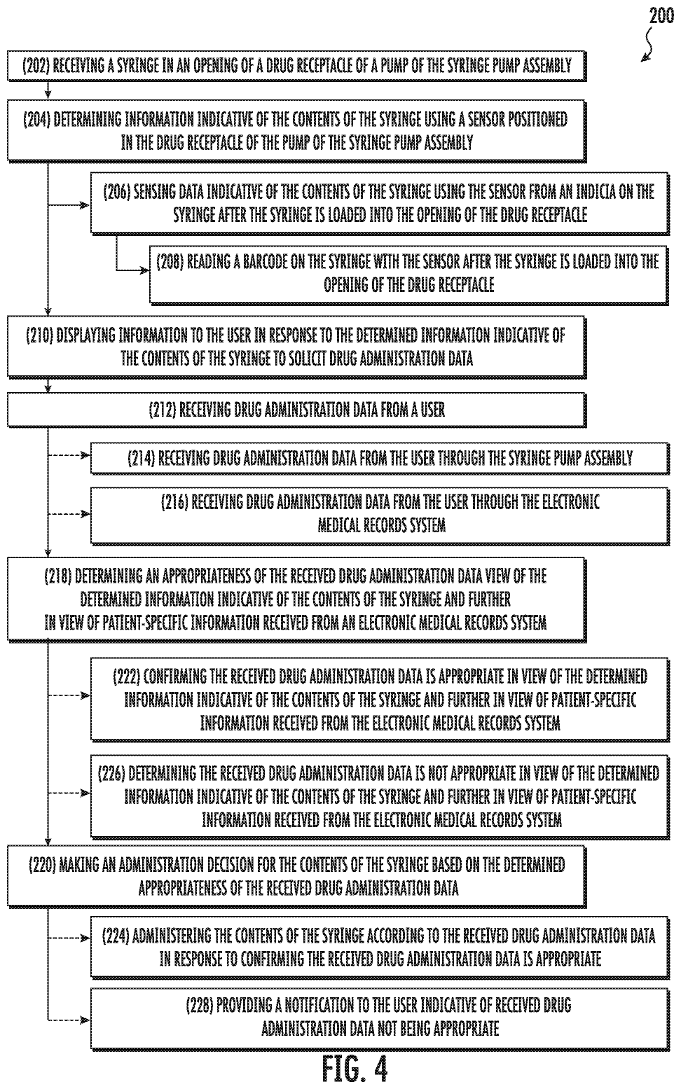

The one or more memory device(s) 106B can store information accessible by the one or more processor(s) 106A, including computer-readable instructions 106C that can be executed by the one or more processor(s) 106A. The instructions 106C can be any set of instructions that when executed by the one or more processor(s) 106A, cause the one or more processor(s) 106A to perform operations (such as the method steps discussed below with reference to FIG. 4). In some embodiments, the instructions 106C can be executed by the one or more processor(s) 106A to cause the one or more processor(s) 106A to perform operations, such as any of the operations and functions for which the controller 106 are configured to perform (e.g., the method 200 steps discussed below), as described herein, and/or any other operations or functions of the one or more computing device(s) 106. The instructions 106C can be software written in any suitable programming language or can be implemented in hardware. Additionally, and/or alternatively, the instructions 106C can be executed in logically and/or virtually separate threads on processor(s) 106A. The memory device(s) 106B can further store data 106D that can be accessed by the processor(s) 106A. For example, the data 106D can include data indicative of power flows, data indicative of engine/aircraft operating conditions, and/or any other data and/or information described herein.

The computing device(s) 106 can also include a network interface 106E used to communicate, for example, with the other components of the syringe pump assembly 100. For example, in the embodiment depicted, the controller 106 is operably coupled to the pump 102 through the network interface 106E, such that the controller 106 may receive data from the pump 102 (e.g., drug administration data from the one or more inputs 114 of the pump 102; sensed data) and further may provide commands to the pump 102 (e.g., drug administration commands).

The network interface 106E can include any suitable components for interfacing with one or more network(s), including for example, transmitters, receivers, ports, controllers, antennas, and/or other suitable components.

The technology discussed herein makes reference to computer-based systems and actions taken by and information sent to and from computer-based systems. One of ordinary skill in the art will recognize that the inherent flexibility of computer-based systems allows for a great variety of possible configurations, combinations, and divisions of tasks and functionality between and among components. For instance, processes discussed herein can be implemented using a single computing device or multiple computing devices working in combination. Databases, memory, instructions, and applications can be implemented on a single system or distributed across multiple systems. Distributed components can operate sequentially or in parallel.

Moreover, the syringe pump assembly 100 is operable with an electronic medical records (EMR) system 122. Specifically, for the embodiment shown, the syringe pump assembly 100 is in communication with the EMR system 122 through the controller 106. In such a manner, it will be appreciated that the syringe pump assembly 100 may communicate information with the EMR system 122. For example, the syringe pump assembly 100 and the EMR system 122 may communicate between one another: proposed drug administration data for a particular patient; confirmation that the proposed drug administration data for the particular patient is acceptable/ not unacceptable; revised drug administration data; error messages; warning messages; requests for additional information; etc.

For the embodiment shown, the EMR system 122 includes a display 123 and an input 125. The display may communicate information to a user/administrator, such as patient information, available drug information, etc. The input 125 may receive one or more user inputs related to the patient and/or drug administration. Additionally, or alternatively, the pump 102 may receive one or more user inputs related to the patient and/or drug administration.

As will be appreciated, the EMR system 122 may be any suitable EMR system. As such, the EMR system 122 may generally include a patient database, with the patent database including relevant information for a plurality of patients, the relevant information including, e.g., a medical history, a list of relevant medical conditions, a list of allergies, treatment protocols, physical attributes of the patient (e.g., height, weight, age, sex, etc.), and other relevant information.

Further, it will be appreciated that the EMR system 122 may include one or more of the components described above with reference to the controller 106 (e.g., one or more processors, memory including data (such as a patient database) and instructions, a network interface, etc.), as well as one or more peripherals in addition to the display 123 and input 125.

In addition, the EMR system 122, the controller 106, or both may include a database of appropriate treatment protocols. The EMR system 122, the controller 106, or both may thus be capable of receiving proposed drug administration data for a particular patient, and compare the proposed drug administration data to the appropriate treatment protocols based on/in view of the patient-specific information available in the patient database of the EMR system 122.

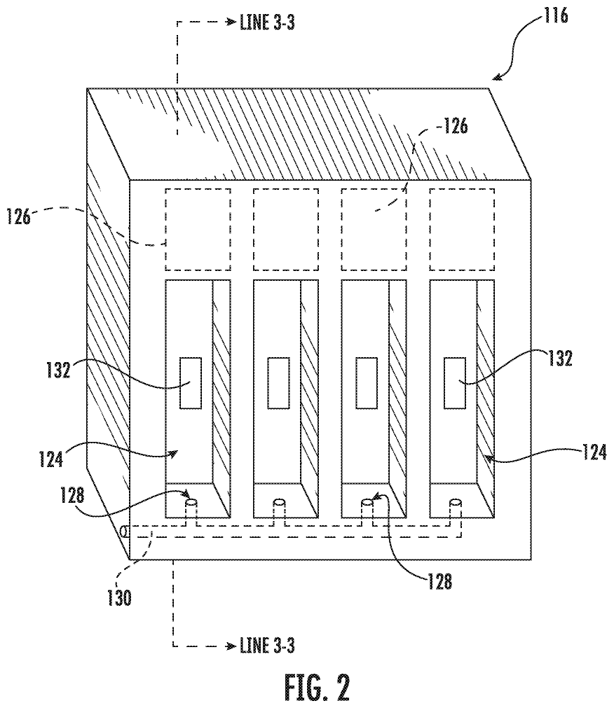

Referring now to FIG. 2, a schematic, perspective view of the drug receptacle 116 of the pump 102 is depicted. The drug receptacle 116 is depicted without any syringes for clarity. The drug receptacle 116 generally defines one or more openings 124 for receiving a syringe. Notably, as used herein, the term “syringe” is used broadly to refer to a container including a drug and is not limited to any specific container type.

Specifically, for the embodiment shown, the drug receptacle 116 includes four openings 124. However, in other embodiments, the drug receptacle 116 may include any other suitable number of openings 124.

In addition, the drug receptacle 116 includes one or more pumps 126 (depicted in phantom) for depressing the syringe and forcing the contents of the syringe through an outlet 128 and into and through an outlet line 130. The outlet line 130 may be fluidly coupled to the output 104, and the line 118 and IV bag 120 of the output 104, depicted in FIG. 1. For the embodiment depicted, the drug receptacle 116 includes a pump 126 for each opening 124.

Moreover, the drug receptacle 116 includes a plurality of sensors 132, each sensor 132 positioned to sense data from a syringe within a respective opening 124. For the embodiment depicted, the sensors 132 are optical sensors, such as barcode readers, QR code readers, written word readers, etc. positioned within/oriented through a wall of the drug receptacle 116 defining a respective opening 124. It will be appreciated, however, that in other embodiments, the sensors 132 may additionally, or alternatively, include any other suitable type of sensor, such as an RFID sensor, a tactile sensor, an electromagnetic sensor, or any other type of sensor capable of sensing or otherwise receiving data from a drug installed therein.

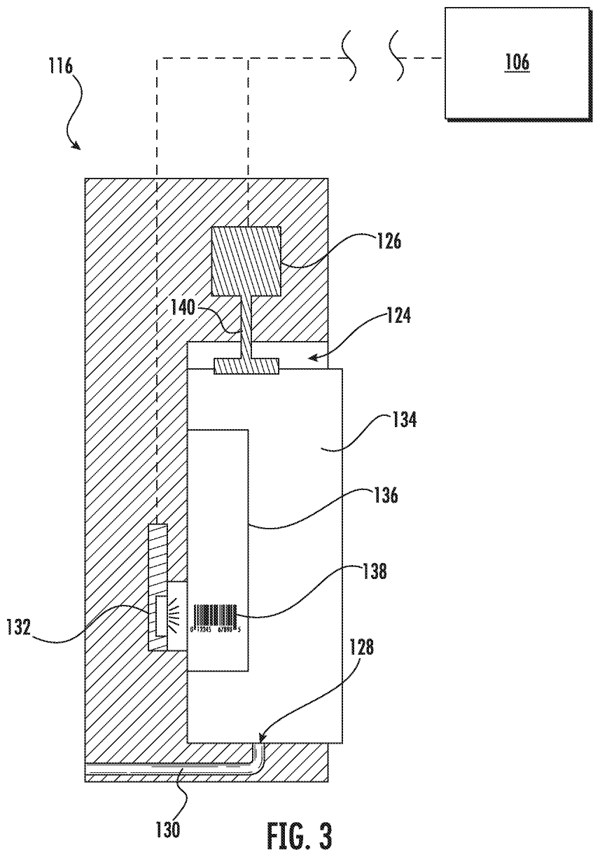

Referring now to FIG. 3, providing a cross-sectional, schematic view of the drug receptacle 116 through Line 3-3 of FIG. 2, a syringe 134 is depicted positioned within an opening 124. As is depicted, the syringe 134 includes a label 136, and the label includes an indicia that is representative of the contents of the syringe 134. For the embodiment shown, the indicia is a barcode 138. However, in other embodiments, the indicia may instead be any other suitable indicia, such as a QR code (or variants thereof), written language, etc. Additionally, or alternatively, the syringe 134 may include other means for communicating identifying information, e.g., an RFID tag, a non-visible graphical indicia (e.g., viewable through a UV light optical sensor), etc.

Moreover, as shown, the sensor 132 is positioned to sense the indicia, and more specifically is positioned to view the graphical indicia/barcode 138 and receive information from such graphical indicia/barcode 138.

Further, the pump 126 includes a plunger 140 configured to physically depress the syringe 134 and force the contents of the syringe 134 through the outlet line 130.

It will be appreciated that for the embodiment shown, both the pump 126 and sensor 132 are operably coupled to the controller 106, such that the controller 106 may receive data from the sensors 132 and provide commands to the pumps 126. The controller 106 may be in wired or wireless communication with such components. It will further be appreciated that each of the pumps 126 and sensors 132 of the drug receptacle 116 may be operably coupled to the controller 106.

From the description provided herein, it will be appreciated that the syringe pump assembly 100 may communicate with the EMR system 122. The syringe pump 102 of the syringe pump assembly 100 is equipped with an optical sensor (sensor 132, which for the embodiment depicted is a barcode reader) oriented along a barrel of the syringe chamber/opening 124. The syringe pump assembly 100 may read a barcode 138 on the syringe 134. Once the barcode 138 is read, the appropriate drugs may become available to select on a screen of the EMR system 122. Via the EMR system 122 the practitioner can select a drug dose and dictate the rate of administration (collectively, “administration data”).

In addition, the syringe pump assembly 100 may elicit an alert to, e.g., the user in the event that an unscheduled and/or inappropriate drug is inserted and sensed. For example, the syringe pump assembly 100 may determine the sensed drug is unscheduled or inappropriate based at least in part on the patient specific information available through the EMR system 122, the procedure being performed, etc. Additionally, or alternatively, the syringe pump assembly 100 may elicit an alert to, e.g., the user in the event a drug scheduled for administration is not present and/or not fully administered at the start of a procedure.

The syringe pump assembly 100 workflow leverages embedded information in the EMR system 122 (e.g., known allergies and weight-dose checking) to confirm the appropriateness of the administration data, and once confirmed, execute the administration data automatically. Such a process may avoid some human error and also can be programed to prevent administration of drugs that should not be used for a specific clinical encounter (e.g., use of paralytic in unsafe environment). Software modification, profiles, and safeguards may be customized by EMR vendors or end users.

Referring now to FIG. 4, a method 200 is provided for operating a syringe pump assembly. The method 200 may be utilized with one or more of the exemplary syringe pump assemblies discussed above with reference to FIGS. 1 through 3.

As shown in FIG. 4, the method 200 includes at (202) receiving a syringe in an opening of a drug receptacle of a pump of the syringe pump assembly, and at (204) determining information indicative of the contents of the syringe using a sensor positioned in the drug receptacle of the pump of the syringe pump assembly. For the exemplary aspect depicted, determining information indicative of the contents of the syringe using the sensor at (204) includes at (206) sensing data indicative of the contents of the syringe using the sensor from an indicia on the syringe after the syringe is loaded into the opening of the drug receptacle. More specifically, for the exemplary aspect depicted, sensing data indicative of the contents of the syringe using the sensor from the indicia on the syringe at (206) includes at (208) reading a barcode on the syringe with the sensor after the syringe is loaded into the opening of the drug receptacle.

For example, it will be appreciated that in certain exemplary embodiments, the sensor may be positioned within the opening of the drug receptacle, or may otherwise define a line of sight to the opening of the drug receptacle (e.g., may be recessed back from the opening and be oriented through a wall defining the opening). Notably, in such a manner it will be appreciated that the sensor may be an optical sensor. It will be appreciated, however, that in other exemplary aspects, the sensor may be configured in an other suitable matter (and the indicia on the syringe may be configured as any other suitable indicia) for sensing data indicative of the contents of the syringe.

The exemplary aspect of the method 200 depicted in FIG. 4 further includes at (210) displaying information to the user in response to the determined information indicative of the contents of the syringe to solicit drug administration data. The display of information may be on a screen of the syringe pump assembly or on a screen (or other peripheral) of a computing device in communication with the syringe pump assembly.

Referring still to the exemplary method 200 of FIG. 4, the method 200 includes at (212) receiving drug administration data from a user. In certain exemplary aspects, receiving drug administration data from the user at (212) may include at (214) receiving drug administration data from the user through the syringe pump assembly, or alternatively may include at (216) receiving drug administration data from the user through the electronic medical records system. The drug administration data may include dosage information and rate of administration data.

The exemplary method 200 further includes at (218) determining an appropriateness of the received drug administration data view of the determined information indicative of the contents of the syringe and further in view of patient-specific information received from an electronic medical records system.

Notably, the patient-specific information may include, e.g., known allergies, weight-dose check information, other medication the patient is using, or a combination thereof. In such a manner, the method 200 at (218) may compare the drug administration data to the patient-specific information to ensure, e.g., the patient is not allergic to the contents of the syringe, that the contents of the syringe do not conflict with any other medications the patient is on, that the dosage and rate of administration for the contents of the syringe is appropriate for the patient based on the patient's size, age, or both, etc.

The method 200 further includes at (220) making an administration decision for the contents of the syringe based on the determined appropriateness of the received drug administration data.

For example, in certain exemplary aspects, determining the appropriateness of the received drug administration data at (218) includes at (222) confirming the received drug administration data is appropriate in view of the determined information indicative of the contents of the syringe and further in view of patient-specific information received from the electronic medical records system. In such a case, making the administration decision for the contents of the syringe at (220) includes at (224) administering the contents of the syringe according to the received drug administration data in response to confirming the received drug administration data is appropriate.

For example, in other exemplary aspects, determining the appropriateness of the received drug administration data at (218) includes at (226) determining the received drug administration data is not appropriate in view of the determined information indicative of the contents of the syringe and further in view of patient-specific information received from the electronic medical records system. In such a case, making the administration decision for the contents of the syringe at (220) includes at (228) providing a notification to the user indicative of received drug administration data not being appropriate. Such notification may be an audible notification, a visual notification, a graphical notification, etc.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal language of the claims.

Further aspects of the invention are provided by the subject matter of the following clauses:

A method for operating a syringe pump assembly comprising: receiving a syringe in an opening of a drug receptacle of a pump of the syringe pump assembly; determining information indicative of the contents of the syringe using a sensor positioned in the drug receptacle of the pump of the syringe pump assembly; receiving drug administration data from a user; determining an appropriateness of the received drug administration data view of the determined information indicative of the contents of the syringe and further in view of patient-specific information received from an electronic medical records system; and making an administration decision for the contents of the syringe based on the determined appropriateness of the received drug administration data.

The method of one or more of these clauses, wherein determining the appropriateness of the received drug administration data comprises confirming the received drug administration data is appropriate in view of the determined information indicative of the contents of the syringe and further in view of patient-specific information received from the electronic medical records system, and wherein making the administration decision for the contents of the syringe comprises administering the contents of the syringe according to the received drug administration data in response to confirming the received drug administration data is appropriate.

The method of one or more of these clauses, wherein determining the appropriateness of the received drug administration data comprises determining the received drug administration data is not appropriate in view of the determined information indicative of the contents of the syringe and further in view of patient-specific information received from the electronic medical records system, and wherein making the administration decision for the contents of the syringe comprises providing a notification to the user indicative of received drug administration data not being appropriate.

The method of one or more of these clauses, wherein receiving drug administration data from the user comprises receiving drug administration data from the user through the syringe pump assembly.

The method of one or more of these clauses, wherein receiving drug administration data from the user comprises receiving drug administration data from the user through the electronic medical records system.

The method of one or more of these clauses, wherein the patient-specific information includes known allergies, weight-dose check information, or both.

The method of one or more of these clauses, wherein determining information indicative of the contents of the syringe using the sensor comprises sensing data indicative of the contents of the syringe using the sensor from an indicia on the syringe after the syringe is loaded into the opening of the drug receptacle.

The method of one or more of these clauses, wherein sensing data indicative of the contents of the syringe using the sensor from the indicia on the syringe comprises reading a barcode on the syringe with the sensor after the syringe is loaded into the opening of the drug receptacle.

The method of one or more of these clauses, wherein the sensor is positioned within the opening of the drug receptacle, or is to define a line of sight to the opening of the drug receptacle.

The method of one or more of these clauses, wherein the sensor is an optical sensor.

The method of one or more of these clauses, further comprising: displaying information to the user in response to the determined information indicative of the contents of the syringe to solicit drug administration data.

The method of one or more of these clauses, wherein the drug administration data includes dosage information and rate of administration data.

A syringe pump assembly comprising: a drug receptacle defining an opening for receiving a syringe; a pump operable with the opening for administering the contents of the syringe when the syringe is positioned within the opening; a sensor configured to sense data indicative of information indicative of a content of the syringe when the syringe is positioned within the opening; and a controller operable with the pump and the sensor, the controller configured to: receive the sensed data indicative of a content of the syringe; receiving drug administration data from a user; determine an appropriateness of the received drug administration data at least in part in view of the sensed data indicative of the contents of the syringe; and make an administration decision for the contents of the syringe based on the determined appropriateness of the received drug administration data.

The syringe pump assembly of one or more of these clauses, wherein the sensor is a barcode reader, and wherein the sensed data indicative of the contents of the syringe is a barcode.

The syringe pump assembly of one or more of these clauses, wherein the sensor is positioned within the opening of the drug receptacle.

The syringe pump assembly of one or more of these clauses, wherein the opening is a first opening, wherein the pump is a first pump, wherein the sensor is a first sensor, wherein the drug receptacle further defines a second opening for receiving a second syringe, and wherein the syringe pump assembly further comprises: a second pump operable with the second opening for administering the contents of the second syringe when the second syringe is positioned within the second opening; a second sensor configured to sense data indicative of a content of the second syringe when the syringe is positioned within the second opening.

The syringe pump assembly of one or more of these clauses, wherein the controller is further configured to be in communication with an electronic medical records system, and wherein in determining the appropriateness of the received drug administration data at least in part in view of the sensed data indicative of the contents of the syringe the controller is further configured to determine the appropriateness of the received drug administration data at least in part in view of the sensed data indicative of the contents of the syringe and patient-specific information received from the electronic medical records system.

The syringe pump assembly of one or more of these clauses, wherein in determining the appropriateness of the received drug administration data the controller is further configured to confirm the received drug administration data is appropriate in view of the sensed data indicative of the contents of the syringe and further in view of patient-specific information received from the electronic medical records system, and wherein in making the administration decision for the contents of the syringe the controller is further configured to administer the contents of the syringe according to the received drug administration data in response to confirming the received drug administration data is appropriate.

The syringe pump assembly of one or more of these clauses, wherein in determining the appropriateness of the received drug administration data the controller is further configured to determine the received drug administration data is not appropriate in view of the sensed data indicative of the contents of the syringe and further in view of patient-specific information received from the electronic medical records system, and wherein in making the administration decision for the contents of the syringe the controller is configured to provide a notification to the user indicative of received drug administration data not being appropriate.

The syringe pump assembly of one or more of these clauses, wherein the drug administration data includes dosage information and rate of administration data.

What is claimed is:

1. A method for operating a syringe pump assembly comprising:
receiving a syringe in an opening of a drug receptacle of a pump of the syringe pump assembly;
determining information indicative of the contents of the syringe using a sensor positioned in the drug receptacle of the pump of the syringe pump assembly;
receiving drug administration data from a user;
determining an appropriateness of the received drug administration data view of the determined information indicative of the contents of the syringe and further in view of patient-specific information received from an electronic medical records system; and
making an administration decision for the contents of the syringe based on the determined appropriateness of the received drug administration data.

2. The method of claim 1, wherein determining the appropriateness of the received drug administration data comprises confirming the received drug administration data is appropriate in view of the determined information indicative of the contents of the syringe and further in view of patient-specific information received from the electronic medical records system, and wherein making the administration decision for the contents of the syringe comprises administering the contents of the syringe according to the received drug administration data in response to confirming the received drug administration data is appropriate.

3. The method of claim 1, wherein determining the appropriateness of the received drug administration data comprises determining the received drug administration data is not appropriate in view of the determined information indicative of the contents of the syringe and further in view of patient-specific information received from the electronic medical records system, and wherein making the administration decision for the contents of the syringe comprises providing a notification to the user indicative of received drug administration data not being appropriate.

4. The method of claim 1, wherein receiving drug administration data from the user comprises receiving drug administration data from the user through the syringe pump assembly.

5. The method of claim 1, wherein receiving drug administration data from the user comprises receiving drug administration data from the user through the electronic medical records system.

6. The method of claim 1, wherein the patient-specific information includes known allergies, weight-dose check information, or both.

7. The method of claim 1, wherein determining information indicative of the contents of the syringe using the sensor comprises sensing data indicative of the contents of the syringe using the sensor from an indicia on the syringe after the syringe is loaded into the opening of the drug receptacle.

8. The method of claim 7, wherein sensing data indicative of the contents of the syringe using the sensor from the indicia on the syringe comprises reading a barcode on the syringe with the sensor after the syringe is loaded into the opening of the drug receptacle.

9. The method of claim 7, wherein the sensor is positioned within the opening of the drug receptacle, or is to define a line of sight to the opening of the drug receptacle.

10. The method of claim 7, wherein the sensor is an optical sensor.

11. The method of claim 7, further comprising:
displaying information to the user in response to the determined information indicative of the contents of the syringe to solicit drug administration data.

12. The method of claim 7, wherein the drug administration data includes dosage information and rate of administration data.

13. A syringe pump assembly comprising:
a drug receptacle defining an opening for receiving a syringe;
a pump operable with the opening for administering the contents of the syringe when the syringe is positioned within the opening;
a sensor configured to sense data indicative of information indicative of a content of the syringe when the syringe is positioned within the opening; and
a controller operable with the pump and the sensor, the controller configured to
receive the sensed data indicative of a content of the syringe;
receiving drug administration data from a user;
determine an appropriateness of the received drug administration data at least in part in view of the sensed data indicative of the contents of the syringe; and
make an administration decision for the contents of the syringe based on the determined appropriateness of the received drug administration data.

14. The syringe pump assembly of claim 13, wherein the sensor is a barcode reader, and wherein the sensed data indicative of the contents of the syringe is a barcode.

15. The syringe pump assembly of claim 13, wherein the sensor is positioned within the opening of the drug receptacle.

16. The syringe pump assembly of claim 13, wherein the opening is a first opening, wherein the pump is a first pump, wherein the sensor is a first sensor, wherein the drug receptacle further defines a second opening for receiving a second syringe, and wherein the syringe pump assembly further comprises:
a second pump operable with the second opening for administering the contents of the second syringe when the second syringe is positioned within the second opening;
a second sensor configured to sense data indicative of a content of the second syringe when the syringe is positioned within the second opening.

17. The syringe pump assembly of claim 13, wherein the controller is further configured to be in communication with an electronic medical records system, and wherein in determining the appropriateness of the received drug administration data at least in part in view of the sensed data indicative of the contents of the syringe the controller is further configured to determine the appropriateness of the received drug administration data at least in part in view of the sensed data indicative of the contents of the syringe and patient-specific information received from the electronic medical records system.

18. The syringe pump assembly of claim 13, wherein in determining the appropriateness of the received drug administration data the controller is further configured to confirm the received drug administration data is appropriate in view of the sensed data indicative of the contents of the syringe and further in view of patient-specific information received from the electronic medical records system, and wherein in making the administration decision for the contents of the syringe the controller is further configured to administer the contents of the syringe according to the received drug administration data in response to confirming the received drug administration data is appropriate.

19. The syringe pump assembly of claim 13, wherein in determining the appropriateness of the received drug administration data the controller is further configured to determine the received drug administration data is not appropriate in view of the sensed data indicative of the contents of the syringe and further in view of patient-specific information received from the electronic medical records system, and wherein in making the administration decision for the contents of the syringe the controller is configured to provide a notification to the user indicative of received drug administration data not being appropriate.

20. The syringe pump assembly of claim 13, wherein the drug administration data includes dosage information and rate of administration data.
